(19) United States
(12) Patent Application Publication
(10) Pub. No.: US 2023/0309840 A1
(43) Pub. Date: Oct. 5, 2023

(54) TARGETING ABNORMAL CELL POPULATIONS FOR APOPTOSIS

(71) Applicants: Mark H. KRIETZMAN, Rolling Hill, CA (US); Renz Wolfgang, Rheinfelden (DE)

(72) Inventors: Mark H. Krietzman, Rolling Hills, CA (US); Wolfgang Renz, Rheinfelden (DE)

(21) Appl. No.: 18/023,335

(22) PCT Filed: Aug. 25, 2021
(86) PCT No.: PCT/US2021/047471

§ 371 Date: Feb. 24, 2023

Related U.S. Application Data
(60) Provisional application No. 63/069,998, filed on Aug. 25, 2020.
(60) Provisional application No. 63/070,116, filed on Aug. 25, 2020.

(51) Int. Cl. A61B 5/0205; A61B 5/145; A61M 5/14; A61M 5/168

(52) U.S. Cl. CPC A61B 5/02055; A61B 5/14532; A61M 5/1407; A61M 5/16827; A61M 5/16831; A61M 2205/3337

(57) ABSTRACT

Controllers induce a hypoglycemic condition within a predetermined blood glucose range abnormal cells including senescent and cancerous cells are targeted for apoptosis. One or more controllers are in signal communication with sensors and measure physiological vital signs. Controllers are in signal communication with one or more fluid flow control devices to control delivery of at least insulin and glucose and at least one cocktail containing at least one of a senolytic and chemotherapeutic. The fluid control devices are in signal communication with at least one microprocessor having memory and the one or more physiological sensors, one or more databases or lookup tables and, wherein the controller controls the fluid control devices for at least insulin glucose, and the cocktail to keep blood glucose level (BGL) within a target hypoglycemic range.

### CROSS-REFERENCE TO RELATED APPLICATIONS

The present application is a 371 National Stage of International Patent Application No. PCT/US2021/047471, filed Aug. 25, 2021, which claims priority to U.S. Provisional Patent Application Ser. No. 63/069,998, filed Aug. 25, 2020, entitled METHOD OF TREATMENT FOR PULMONARY INFLAMMATION; and also claims priority to U.S. Provisional Patent Application Ser. No. 63/070,116, filed Aug. 25, 2020, entitled METHOD OF TREATMENT FOR INFLAMMATION, which are hereby incorporated by reference in their entirety.

### FIELD OF THE DISCLOSURE

Systems and methods to control hypoglycemic conditions in a primate during therapeutic treatment.

### BACKGROUND

Abnormal cells include cells such as cancer which if left unchecked grow and are likely to metastasize eventually causing a multitude of health problems and/or death. Another abnormal cell type are senescent cells which are a populations of cells that increase in the tissues and organs during ageing. Senescence cells impact their surrounding cells. In a senescent cell there is a senescence-associated secretory phenotype (SASP). SASP arises due to the increased production and secretion of proteins, which are involved in the generation of low-grade inflammation. This chronic inflammation may also drive surrounding cells to become senescent, cause inflammation related tissue damage, and encourage cancer. It has been suggested that senescent cells can change the tumor microenvironment and facilitate invasion, metastasis, and resistance to therapies. Therapies targeting senescent cells to promote their apoptosis or lysis are hampered with the need to prevent apoptosis of healthy cells. It is therefore a desideratum to reduce and remove the populations of senescent cells.

Cancer cells will, if left unchecked, grow and are likely to metastasize. These proliferating cells have different metabolic requirements from non-proliferating cells. Such cells consume tremendous amounts of glucose and metabolize the majority of the glucose into lactate, even in the presence of oxygen (Warburg, 1924). This phenomenon is referred to as aerobic glycolysis and represents a large metabolic difference between cancer and most normal tissues. Unfortunately, therapeutics that block glucose metabolism also slow glucose uptake in many noncancerous tissues, including the brain because high glucose uptake is not a unique feature of tumors and offers a potential explanation for the relative lack of success in the traditional directly targeting glucose metabolism for cancer treatment. It is therefore a desideratum to reduce and remove the populations of cancer cells by way of the metabolic needs of such cells.

### DISCLOSURE

Disclosed herein are aspects of devices, methods, and systems of maintaining a dynamically controlled hypoglycemic environment to reduce abnormal cell populations in a primate. Some aspects of exemplary implementations include pretreating abnormal cells, which have yet to be identified with a disease state, whereby cellular membranes have increased permeability to therapeutics which promote apoptosis of such cells.

Disclosed herein are aspects of devices, methods and systems of delivering a method of at least one of an effective treatment a supplement to abnormal cell populations, including but not limited to disrupting the cellular membrane of the abnormal cells including cancerous or senescent cell then contacting the cells with an effective amount of at least one senolytic and chemotherapeutic agents which is more likely to cellularly disrupted (cause apoptosis to) abnormal cells then normal cells.

Disclosed herein are aspects of devices, methods, and systems of reducing abnormal cells populations. In some exemplary implementations' aspects include placing abnormal cells in hypoglycemic conditions to increased membrane permeability to at least one of hemolytic, and chemotherapeutic compounds which also can be referred to as “cocktail” or “cocktail components”.

In some instances, one or more cocktail components are within or on the surface of a micelle or liposome and has an affinity for the membrane potential of senescent cells. In some instances, the micelle has an affinity for cells expressing at least one of CDKN2A (p16^{Ink-4a}) and SASP.

In some exemplary implementations aspects include maintaining hypoglycemic conditions in a primate to increased membrane permeability of cells and in particular to abnormal cells to at least one of senolytic, and chemotherapeutic compounds which also can be referred to as “cocktail” or “cocktail components” In some instances the damage to the cancer cells is severe and leads to cell death at a higher rate than mild damage which may lead to senescence and allow for cancer progression or metastasis. In some instances, at least some of the cocktail compounds may be bound in or to a micelle or liposome.

Disclosed herein are aspects of devices, methods and systems for drug development and testing under hypoglycemic conditions. The method further comprising delivering one of a supplement and a pharmaceutically effective dose of chemotherapeutic and/or senolytic agents to a population of abnormal cells in an animal model which have been selectively placed in a controlled hypoglycemic condition. In some cases, the animal model is a primate. In some cases, the animal model is a humanized non-primate, in some cases the animal model is a non-primate mammal.

Disclosed herein are aspects of devices, methods and systems for drug development and testing under hypoglycemic conditions. The method further comprising delivering one of a supplement and a pharmaceutically effective dose of chemotherapeutic and/or senolytic agents to a population of abnormal primate cells in vitro which have been selectively placed in a controlled hypoglycemic condition. Disclosed herein are aspects of devices, methods and systems for drug development and testing under hypoglycemic conditions. The method further comprising delivering one of a supplement and a pharmaceutically effective dose of chemotherapeutic and/or senolytic agents to a population of abnormal primate cells in vivo which have been selectively placed in a controlled hypoglycemic condition.

Disclosed herein are aspects of devices, methods, and systems of delivering one of a supplement and a pharmaceutically effective dose of one or more chemotherapeutic and senolytic (or a cocktail which combines to act as a chemotherapeutic or hemolytic) to a population of cells which are selectively placed in a controlled hypoglycemic condition whereby apoptosis of abnormal cells occurs at a higher rate than apoptosis in normal cells. In some instances, the abnormal cells are cancerous. In some instances, the abnormal cells are senescent.

Disclosed herein are aspects of devices, methods, and systems of delivering a phased or sequenced series of cocktail compounds, forming a pharmaceutically effective dose, to cause apoptosis in cancerous cell populations as at least one of as a therapeutic treatment and a supplement when exposing the abnormal cells to controlled hypoglycemic conditions.

In the above exemplars one or more controllers control hypoglycemic conditions in the test animal or primate via data received from one or more sensor inputs whereby fluid control devices to control the flow of insulin, glucose and optionally additional cocktail component and adjuvants as well as oxygen.

Aspects of the delivery systems, control system and methods disclosed include a control system are configured to maintain a host in a controlled hypoglycemic condition and automatically adjust. Condition to maintain the hypoglycemic condition within a target range of blood glucose levels (BGL) and above a first threshold. In some instances, the system includes logic to raise BGL when the first threshold or a lower second threshold is reached. The thresholds and ranges may be personalized based on collected individual data about a patient prior to treatment with the hypoglycemic method disclosed herein. The system and method include, but are not limited to:
measuring the normal level of the host's insulin pretreatment over a period of between 15 minutes and 48 hours before administering the hypoglycemic protocols.
(ii) Preparing a host specific algorithm in the form of computer code stored in memory and configured to be used in a microprocessor in signal communication with one or more controllers configured to adjust insulin delivery to maintain a hypoglycemic state in the host during therapeutic administration.
(iii) monitoring with sensors in signal communication to the one or more controller one or more of the host's vital signs, including but not limited to, heart rate (HR), blood pressure (BP), electrocardiogram (EKG), electroencephalogram (EEG), oxygen saturation (O₂), galvanic skin response (GSR), skin moisture, pupillary dilation (PD), temperature (T), respiration (R) rate, and blood glucose level (BGL).
(iv) using a controller to meter out at least boluses of insulin (via one or more devices) to place the host in a temporarily hypoglycemic condition at one of a predetermined target range and above a predetermined hypoglycemic threshold.
(v) using a controller to meter out at least boluses of glucose (via one or more devices) to keep the host above a predetermined hypoglycemic threshold.
(vi) The controller(s) configured to use sensor data to control at least one of control the amount and the rate of insulin delivery to keep host to maintain blood glucose levels (BGL) within a defined range corresponding to the target hypoglycemic condition for the host. In some instances, the target hypoglycemic condition for the host is related to or arise from the previously measured levels for that host. In some instances, if measured oxygen saturation is below a predetermined level the controller administers additional to the host.
(vii) optionally one or more alarms are generated via the controller(s) if the controlled hypoglycemic conditions in the host (as measured by the system) are outside of a range selected for the host at a given time during treatment. The alarms may be any form including but not limited to visual, auditory, and haptic. The alarms may at least one of interrupt the insulin delivery, cause glucose to be delivered, cause oxygen to be delivered until vital signs are restored to within the target range.
(viii) Optionally pharmaceutically effective amounts of at least one of an antihistamine and an antiemetic may be administered prior to insulin delivery.
(ix) after the host is in the hypoglycemic condition the devices and systems sequence administration of pharmaceutically effective amounts of one or more chemotherapeutic agents in a pharmaceutically effective dose, under hypoglycemic conditions.
(x) after the host is in the hypoglycemic condition the devices and systems sequence administration of pharmaceutically effective amounts of one or more senolytic agents in a pharmaceutically effective dose, under hypoglycemic conditions.
(xi) optionally measuring at least one of the host's vital signs, including but not limited to ECG, EKC, blood pressure, oxygen saturation, heart rate, galvanic skin response, skin moisture, and temperature response over a period of between 15 minutes and 48 hours before treatment and collecting said data.
(xii) optionally one or more alarms configured in the computer code are configured so that the controller(s) generates an alarm if measured vital sign(s) is outside a predefined range. In some instances, using the previously measured vital sign data to set said range. The alarms may at least one of interrupt the insulin delivery, cause glucose to be delivered, cause oxygen to be delivered until vital signs are restored to within the target range.

In some instances, prior to step (i) at a predetermined interval the host consumes a known quality sugar in a predetermined form with a Glycemic Index (GI) and a known glycemic load (GL). By supplying a consistent food type of a fixed quantity and with a known GI and GL the measurement of the host's innate systems response to the consumed material can be measured via blood glucose monitoring, and used at least in part, as a data point to set the target range for hypoglycemic conditions for that host's treatment.

In some instances, prior to step (iv) at a predetermined interval the host consumes a known quality sugar in a predetermined form with a known GI and GL. By supplying a food type of a fixed quantity and with a known GI and GL the controller can use look up tables (LUT) or refer to prior measurements of the host's consumption of the same GI and GL food and used, at least in part, as a data point when maintaining the target range for hypoglycemic conditions for that host.

Disclosed herein are aspects of devices, methods, compositions of matter and systems to induce a hypoglycemic condition within a predetermined blood glucose range for treating abnormal cells including one or more controllers in signal communication with at least a BGL sensor and may be in signal communication with additional sensors each of which measure an aspect that is physiological and in signal communication with one or more fluid flow control devices to control deliver of at least insulin and glucose and at least one cocktail containing at least one of senolytic and chemotherapeutic components. The fluid control devices are in signal communication with at least one microprocessor having memory and the one or more physiological sensors, one or more databases or lookup tables and, wherein the controller controls the fluid control devices for at least insulin glucose, and the cocktail to keep blood glucose level (BGL) within a target hypoglycemic range for BGL for the patient. In some instances, the controller receives sensor data inputs and adjust the target hypoglycemic BGL range in response to sensory data received. In some instances, sensor data is BGL and one or more of oxygen saturation, heart rate, blood pressure, galvanic skin response, temperature, EEG, ECG, and pupillary response. In some instances, the controller controls the administration of at least one of oxygen and hydrogen.

System and method cocktail components include but are not limited to quercetin (and analogs thereof), enzastaurin, Q10, dasatinib. tocotrienols azithromycin, curcumin, Sirolimus (Rapamycin), Nav-Gal and Navitoclax, Artemisia, hydrogen, oxyhydrogen, vitamin C, Curcuminoid, cannabinoids, γ-Tocotrienols, romidepsin, zolinza (vorinostat), belinostat (also known as PXD101), farydak, panobinostat), ricolinostat (also known as ACY-1215) and citarinostat (also known as ACY-241).

System and method cocktail components include but are not limited Cisplatin and Mitomycin. carboplatin combined with vinorelbine 5-FU and Gemcitabine, Docetaxel, plant alkaloids, antitumor antibiotics, antimetabolites, topoisomerase inhibitors, mitotic inhibitors, nitrosoureas and anthracyclines.

Disclosed herein are aspects of devices, methods, compositions of matter and systems to induce a hypoglycemic condition within a predetermined blood glucose range for treating abnormal cells including one or more controllers in signal communication with at least a BGL sensor and may be in signal communication with additional sensors each of which measure an aspect that is physiological and in signal communication with one or more fluid flow control devices to control deliver of at least insulin and glucose and at least one cocktail containing at least one of senolytic and chemotherapeutic components. The fluid control devices are in signal communication with at least one microprocessor having memory and the one or more physiological sensors, one or more databases or lookup tables and, wherein the controller controls the fluid control devices for at least insulin glucose, and the cocktail to keep blood glucose level (BGL) within a target hypoglycemic range for BGL for the patient. In some instances, the controller receives sensor data inputs and adjust the target hypoglycemic BGL range in response to sensory data received. In some instances, sensor data is BGL and one or more of oxygen saturation, heart rate, blood pressure, galvanic skin response, temperature, EEG, ECG, and pupillary response. In some instances, the active agents in the cocktail are each less than 50% the maximum tolerated dose (MTD). In some instances, the active agents in the cocktail are less than 50% the minimum effective dose (MED) In some instances, the active agents in the cocktail are less than 25% the minimum effective dose (MED) In some instances, the active agents in the cocktail are less than 15% the minimum effective dose (MED).

Disclosed herein are aspects of devices, methods, compositions of matter and systems to reduce abnormal cell populations in a primate including inducing and maintaining a hypoglycemic condition in a primate having abnormal cells within a target BGL range by way of infusion of insulin controlled by a controller; monitoring vital signs of the primate with one or more sensors each of which monitor a physiological aspect of the primate including at least BGL and is in signal communication with the controller; controlling with a controller infusion of a cocktail containing at least one of senolytic and chemotherapeutic components into the primate while the primate is in a hypoglycemic condition; wherein the controller receives data inputs form the sensors and at least in part uses that input data to one of maintain the primate's BGL within a target hypoglycemic BGL range and alter the target hypoglycemic BGL range or lower threshold for the hypoglycemic BGL based on the received sensor data; and. wherein abnormal cells cellular membranes are made more susceptible to the influx of cocktail components by way of the hypoglycemic condition. In some instances, the abnormal cells are one of cancerous and senescent. In some instances, the abnormal cells have more insulin receptors then normal cells. In some instances, the abnormal cells have more transferrin receptors then normal cells. In some instances, the sensor data is one of BGL, oxygen saturation, heart rate, blood pressure, galvanic skin response, temperature, EEG, ECG, and pupillary response. In some instances, the controller raises BGL in the primate by way of infusion of at least glucose to maintain the target hypoglycemic BGL range for the primate. In some instances, the controller administers magnesium before or during administration of glucose.

It is appreciated by those skilled in the art that some of the circuits, components, controllers, modules, and/or devices of the system disclosed in the present application are described as being in signal communication with each other, where signal communication refers to any type of communication and/or connection between the circuits, components, modules, and/or devices that allows a circuit, component, module, and/or device to pass and/or receive signals and/or information from another circuit, component, module, and/or device. The communication and/or connection may be along any signal path between the circuits, components, modules, and/or devices that allows signals and/or information to pass from one circuit, component, module, and/or device to another and includes wireless or wired signal paths. The signal paths may be physical such as, for example, conductive wires, electromagnetic wave guides, attached and/or electromagnetic or mechanically coupled terminals, semi-conductive or dielectric materials or devices, or other similar physical connections or couplings. Additionally, signal paths may be non-physical such as free-space (in the case of electromagnetic propagation) or information paths through digital components where communication information is passed from one circuit, component, module, and/or device to another in varying analog and/or digital formats without passing through a direct electromagnetic connection. These information paths may also include analog-to-digital conversions (“ADC”), digital-to-analog (“DAC”) conversions, data transformations such as, for example, fast Fourier transforms (“FFTs”), time-to-frequency conversations, frequency-to-time conversions, database mapping, signal processing steps, coding, modulations, demodulations, etc. The controller devices and smart devices disclosed herein operate with memory and processors whereby code is executed during processes to transform data, the computing devices run on a processor (such as, for example, controller or other processor that is not shown) which may include a central processing unit (“CPU”), digital signal processor (“DSP”), application specific integrated circuit (“ASIC”), field programmable gate array (“FPGA”), microprocessor, etc. Alternatively, portions DCA devices may also be or include hardware devices such as logic circuitry, a CPU, a DSP, ASIC, FPGA, etc. and may include hardware and software capable of receiving and sending information

### FIGURES

The disclosure may be better understood by referring to the following figures. The components in the figures are not necessarily to scale, emphasis instead being placed upon illustrating the principles of the invention. In the figures, like reference numerals designate corresponding parts throughout the different views.

FIG. 1 is a diagram of a host with sensor.

FIG. 2 is a diagram of a system overview

FIG. 3 is a flow diagram for control of the delivery system.

FIG. 4 illustrates aspects of a logic of the system logic.

FIG. 5 shows a nonexclusive list of chemical structures of analogs of Quercetin.

FIG. 6 shows chemical structure of states of Co-enzyme Q10.

FIG. 7 Shows the chemical structure of Curcumin.

FIG. 8 shows a nonexclusive list of curcumin analogs.

FIGS. 9-15 are tables showing sequenced administration of hypoglycemic condition and infusion of therapeutic compounds.

All descriptions and callouts in the Figures and all content of any referenced citation are hereby incorporated by this reference as if fully set forth herein.

### FURTHER DISCLOSURE

The compositions disclosed herein can be included in a pharmaceutical or nutraceutical composition together with additional active agents, carriers, vehicles, excipients, or auxiliary agents identifiable by a person skilled in the art upon reading of the present disclosure, and such compositions are within the scope of this disclosure. All publications cited herein are hereby incorporated by reference as if fully set forth herein.

The pharmaceutical or nutraceutical compositions preferably comprise at least one pharmaceutically acceptable carrier. In such pharmaceutical compositions, the compositions disclosed herein form the “active compound,” also referred to as the “active agent.” As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds and/or adjuvants can also be incorporated into the compositions. A pharmaceutical composition is formulated to be compatible with its intended route of administration.

Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, primate, dog, cat, and rabbit) and most preferably a human. Administration by inhalation, the gas or gases are delivered orally.

As used herein a “primate host” is defined to include a monkey, baboon, chimpanzee, gorilla, and a human. Nonhuman primates are appreciated to themselves be susceptible to infection by retroviruses and in particular immunodeficiency viruses and represent well-established animal models as to human response with an appreciation that physiological differences often require different doses in milligrams per kilogram for a nonhuman primate animal model relative to a human.

Administration” and “treatment,” as it applies to an animal, human, experimental subject, cell, tissue, organ, or biological fluid, refers to contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. “Administration” and “treatment” also means in vitro and ex vivo treatments, e.g., of a cell, by a reagent, diagnostic, binding compound, or by another cell. The term “subject” includes any organism, preferably an animal, more preferably a mammal (e.g., rat, mouse, primate, dog, cat, and rabbit) and most preferably a human.

A pharmaceutically effective dose (ED) or effective concentration (EC) is a dose or concentration of an element (such as hydrogen), a phytochemical, compound ore drug that produces a biological response. The term effective dose is used when measurements are taken in vivo, while the term effective concentration is used when the measurements are taken in vitro. This is generally defined by the range between the minimum effective dose (MED) and the maximum tolerated dose (MTD). The MED is defined as the lowest dose level of a pharmaceutical product that provides a clinically significant response in average efficacy, which is also statistically significantly superior to the response provided by the placebo. Similarly, the MTD is the highest possible but still tolerable dose level with respect to a pre-specified clinical limiting toxicity. In general, these limits refer to the average patient population.

Cell senescence can be associated with a progressive and marked increased rate of glucose metabolism through glycolysis. Senescent cells display widespread changes in chromatin structure (referred to as senescence associated heterochromatin foci, SAHF) and gene expression profiles, which lead to highly active cellular metabolism and secretion of cytokines (TGF-β, IL-1a, -1β and -6), chemokines (IL-8, CXCL1), growth factors (FGF, HGF) and proteases (MMP-1, -3, and -13), collectively defined as senescence associated secretory phenotypes (SASP). SASP is a key factor that leads to the increase in senescence of populations of normal cells in proximity to senescent cells.

Plasma glucose levels are maintained within a narrow range by the pancreatic hormone's glucagon and insulin. A normal level threshold for BGL is about 140 mg/dL Hypoglycemia, is general a BGL of below 50 mg/dl in a non-diabetic and it triggers secretion of glucagon by pancreatic α cells, which promotes glycogenolysis and gluconeogenesis in the liver, and lipolysis in adipose tissue. On the other hand, hyperglycemia triggers secretion of insulin from pancreatic β cells, which promotes glucose uptake for energy production and anabolic processes such as glycogen synthesis and lipogenesis in the liver, muscles, and adipose tissue.

We have observed that controlled hypoglycemic conditions applied to patients via an infusion of insulin during combined with administration of a cocktail is associated with preferential reduction in tumor size and/or abnormal cell markers. The term cocktail refers to one or more of chemotherapeutic, senolytic and therapeutic agents which may include supplements and adjuvants. In some instances, the administration of the cocktail during controlled hypoglycemic conditions results in higher intercellular concentrations of cocktail therapeutics in abnormal cells which metabolize a higher percentage of the cocktail or components of the cocktail then normal cells. Many effective chemotherapeutic and senolytics when administered at a MED without hypoglycemic conditions approach of exceed their MTD. Our controlled hypoglycemic conditions are configured to reduce the MED whereby the cocktail is administered at below the MTD for cocktail components and can achieve effective therapeutic doses.

The system hardware, software, microprocessors, and controllers are configured to adjust the administration (rate and quantity) of insulin to maintain controlled hypoglycemic conditions for an individual patient based on previously collected patient data. The system hardware, software, microprocessors, and controllers may set off alarms if blood glucose is outside a patients predetermined range. The system hardware, software, microprocessors, and controllers are configured to control the infusion of glucose (rate and quantity), oxygen (rate and quantity) and cocktail components (rate and quantity of each) and set off alarms if measured blood glucose and/or vital signs are outside predetermined ranges or levels.

The system hardware, software, microprocessors, and controllers are configured to adjust the insulin administration to maintain controlled hypoglycemic conditions for an individual patient based on previously collected patient data which is used to define an individual target range for and define at least a first BGL lower threshold for that individual. In some instances that data collected is also sued to define a second lower BGL (also known as an alarm level) for that individual.

The system hardware, software, microprocessors, and controllers may override the target for blood glucose target range or first or second threshold levels of a patient based on one or more inputs of sensor data. The microprocessor compares the patient sensor data being collected in real time during hypoglycemic conditions with one or more of a look up table based on human physiology, a look up table (LUT) based on measurements of the patient made prior to treatment, threshold level preset in a decisioning module, and if one or more sensor measurements exceed a risk level a target range or threshold limit may be altered and the controller will then administer an effective amount of insulin or glucose (for example) to raise or lower the BGL to the revised or altered target range or above a revised threshold. The system can override the continuation of insulin administration or reduce the amount given. The system can add glucose to the patient, the system can add oxygen to the patient and the system can adjust infusion of cocktail components to the patient. In some instances, an alarm will be set-off if the sensor data exceeds a threshold. The previous collection of patient data may be over 15 minutes or more but preferably over several hours or over a day.

A simplified overview is that an in vitro tissue culture of normal and abnormal cells is given a bolus of insulin, the cellular insulin receptors (IR) activate and open up expecting sugar which abnormal cells (both senescent and cancer cells metabolize at a higher rate) instead the insulin is followed by cocktail components. In some instances, at least some abnormal cells have less selective ion channels activate by ligands such as insulin and therefore will allow for a higher concentration of cocktail components to enter the abnormal cell then normal cells.

A simplified overview is that an in vivo animal model having both normal and abnormal cells is given a bolus of insulin, the cellular insulin receptors (IR) activate expecting sugar which abnormal cells (both senescent and cancer cells metabolize at a higher rate) instead the insulin is followed by cocktail components. In some instances, at least some abnormal cells have less selective ion channels activate by ligands such as insulin and therefore will allow for a higher concentration of cocktail components to enter the abnormal cell then normal cells.

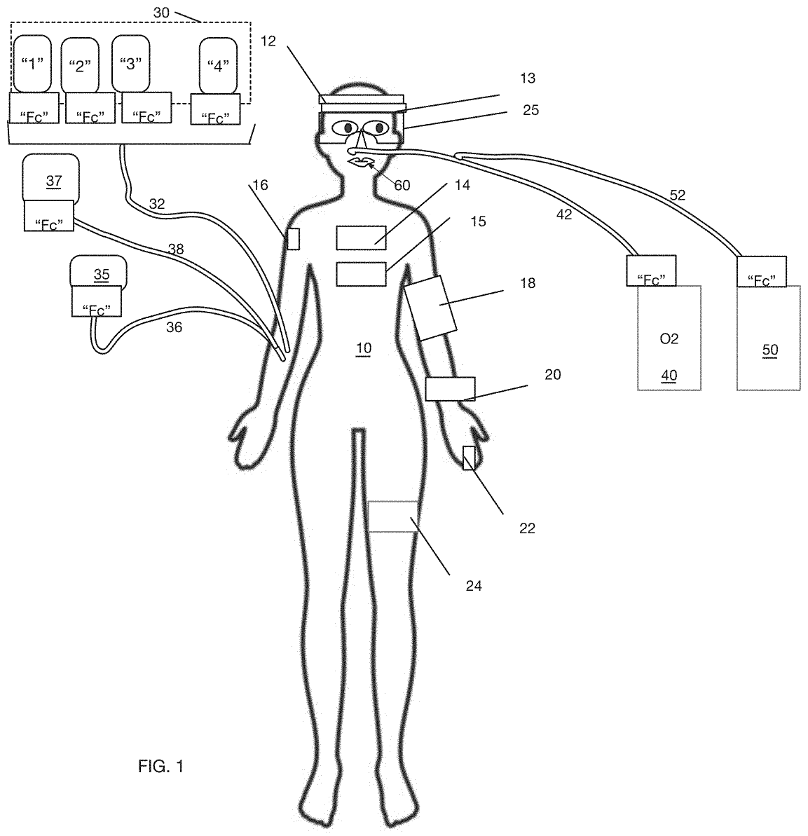

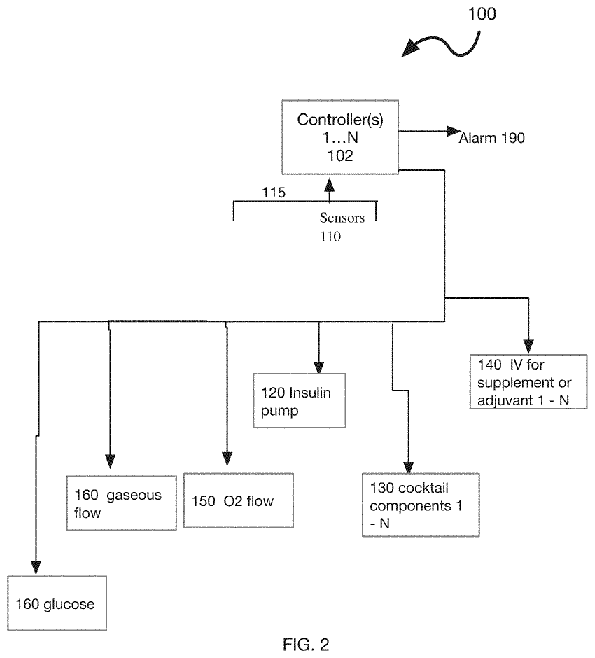

In another simplified overview set forth in FIGS. 1 and 2 in which a primate 10 is connected to sensors 110 which are in signal communication with a controller 102. The primate is connected to or measured by a multitude of sensors 110. Including but not limited to sensors to measure temperature 12, EEC 13, EKG 14, galvanic skin response (which measured sweatiness) 15, blood glucose levels (BGL) 16, blood pressure 18, heart rate 20, oxygen saturation 22 and additional measurements such as skin moisture which may include cortisol level measurements 24. Pupillary response is measured by machine vision optical system 25. Cocktail components 30 (1-N) each in a containment vessel are connected to the patient or host via fluid pathways and the fluid pathways each have a flow control device “fc” to start, stop and regulate the fluid flow, flow control devices include but are not limited to remotely controlled syringe pumps, peristaltic IV pumps, piston driven pumps and valves. Fluid control devices (“fc”) are in signal communication with one or more controllers wherein the fluid flow rate is controlled in response to microprocessor control which in turn is based at least in part on sensor data received and analyzed by the system processors.

The cocktails components are connected to the patient via a fluid communication pathway 32. An insulin source 35 in a containment vessel has a flow control device “fc” in signal communication with a controller and is connected to the patient via a fluid communication pathway 36. A glucose source 37 in a containment vessel has a flow control device “fc” in signal communication with a controller and is connected to the patient via a fluid communication pathway 38. An oxygen source 40 in a containment vessel has a flow control device “fc” in signal communication with a controller and is connected to the patient via a fluid communication pathway 42. A second gaseous fluid source 50 in a containment vessel has a flow control device “fc” in signal communication with a controller and is connected to the patient via a fluid communication pathway 52. The second gaseous fluid source includes but is not limited to hydrogen, oxyhydrogen, and vaporized or atomized cannabinoids. Oral ingestion via the mouth 60 may be an alternative for some of the cocktail components or optional compounds.

A control system overview 100 is a simplified diagram showing controllers 102 which are in signal communication 115 with the sensor 110 outputs. The controller processes the data from the sensors and decisions, based on LUTs, predetermined ranges for a patient and threshold levels to control insulin rate of administration and quantity. The controller also controls the administration of cocktail components 130. The controller 102 also controls the administration of supplement or adjuvant components 140. The controller 102 also controls the administration of oxygen 150. The controller also controls the flow of gaseous fluids 160 such as hydrogen, oxyhydrogen and/or cannabinoids. The controller 102 also controls the administration of glucose 37. The controller also triggers or sets alarms 190 for out of threshold or range measurements. Normally when blood glucose level reach less than 30 mg/dl the system will administer a controlled release of glucose, at a rate based on sensor data, and within a predetermined first threshold (or safe limit). The system is configured for the individual and to avoid a significant glucose deficit which may impair brain function. In those instances, wherein levels fall below a second threshold which is below the first threshold alarms 190 will signal the and the system can administer magnesium to allow for an increased rate of glucose infusion. If glucose is infused too fast physiological stress is created in the vein and cramping. By adding magnesium, a higher flow of glucose can be used to restore the patient above the second threshold. Post treatment after patient stabilized and has BGL above a premeasured and predetermined base level (generally about 15-20 minutes) an oral infusion of at least 250 cc Glucose 40% and also a glucose-based fluid or juice such Coke and/or Apple juice at 500 cc orally helps the patient to maintain blood glucose levels.

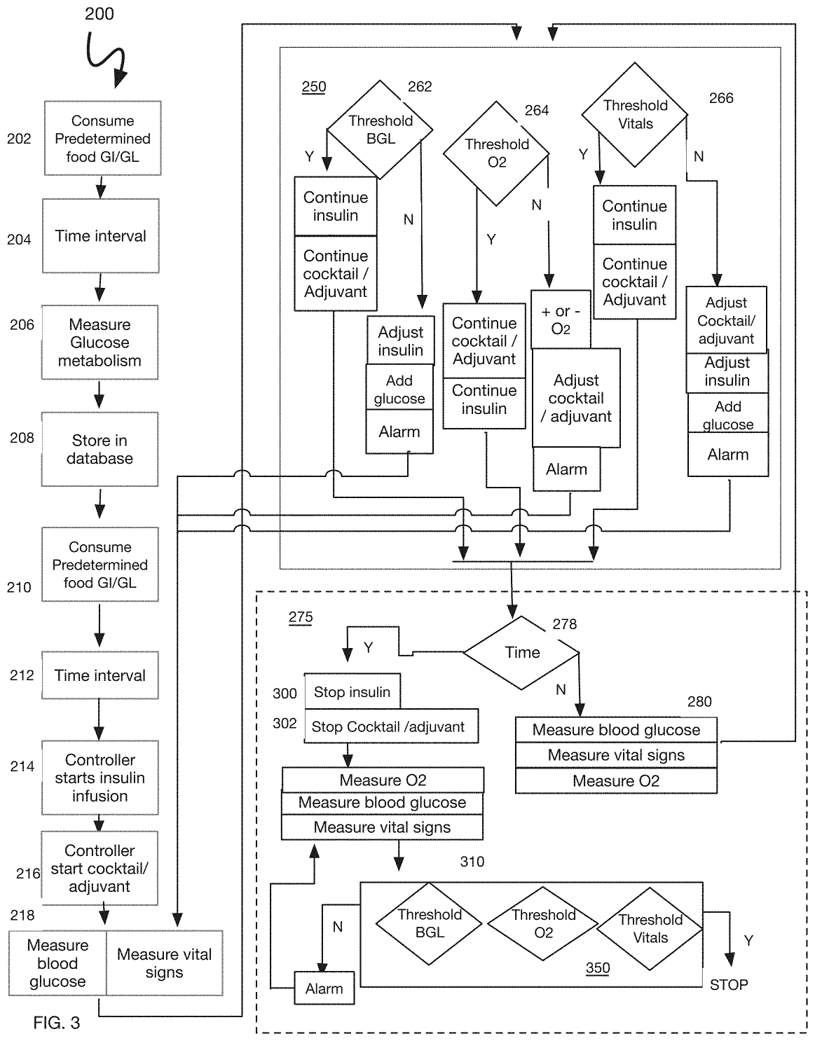

FIG. 3 illustrates an overview of some aspects of the operational flow of the control system 200. Baseline or nominal values for a patient are collected by way of using a predetermined quantity of a known quality sugar in a predetermined form with a known GL 202. During a time, interval 204 the patient's blood glucose levels are measured 206 by way of a blood glucose monitoring device (which are known in the art) and the measurements are collected and stored in a database 208. Prior to treating the patient with the disclosed cocktail under hypoglycemic conditions, the patient consumes once again the predetermined quantity of a known quality sugar in a predetermined form with a known GL 210. A time interval 212 will pass after ingestion of the passes after ingestion of the known quality sugar in a predetermined form with a known GL 210 and the previously measured metabolism by the patient of the known quality sugar in a predetermined form with a known GL 202 is collected and can be used to set a hypoglycemic target range that is individualize for the patient. In some instances, the target range for example may be below 54 mg/dl and above 42 mg/dl and with a lower threshold of 40 mg/dl over a predetermined amount of time. In other instances, the target range for example may be below 45 mg/dl and above 38 mg/dl and with a lower threshold of 35 mg/dl over a predetermined amount of time. Based on the metabolic data collected from the patient prior to treatment the controller can individualize the response to allow a lower threshold based on the time a patient can be at that lower threshold before changing the infusion rate of insulin or adding glucose. The system is predictive and can use the slope of the curve of BGL during treatment to reduce insulin or add glucose prior to the patient falling below a lower threshold. In general, 30 mg/dl is a second threshold that should not be maintained, and the patient should not drop below that threshold. However, based on sensor data that level may be raised for a patient.

The controller starts the infusion of insulin 214, the controller starts the infusion of cocktail and/or adjuvant 216 and the sensors 110 measure the patient's GBL and measures vital signs 218. The skilled artisan or those of ordinary skill in the art will recognize that the sequence of infusing cocktail components, adjuvants, and the like before insulin or vice versa and/or any time gap between the infusions are variations of the disclosed process which are within the scope of this disclosure. A monitoring module 250 receives the measurements and the controller decisions if threshold levels are met for one or more of blood glucose levels (BGL) 262, oxygen 264 and vitals 266. If all threshold levels being monitored are met the then the controller continues the insulin infusion and cocktail/adjuvant infusions, and the system goes on to the timer module 275. If the BGL threshold 262 is not met then the controller will one or more of adjust insulin infusion, add glucose and alert via an alarm. If the O₂ threshold 264 is not met then the controller will one or more of adjust flow rate of the O₂ 150 delivered to patient, adjust one or more cocktail components 130, adjust infusion of one or more supplement/adjuvant components 140 flow rates, adjust other gaseous flow 160 and alert via an alarm. If the vitals threshold 266 is not met then the controller will one or more of adjust insulin infusion, adjust cocktail and/or adjuvant components infusion rates, add glucose and alert via an alarm. If threshold were not met then the sensors 110 measurements of the patient's blood glucose levels and measure of vital signs 218 are processed by the controller and the controller in the monitoring module 250 as described above decisions if the threshold are been met the monitoring and adjustments repeat.

In another exemplar, prior to treating the patient with the disclosed cocktail under hypoglycemic conditions, the patient consumes once again the predetermined quantity of a known quality sugar in a predetermined form with a known GL 210. A time interval 212 will pass after ingestion of the passes after ingestion of the known quality sugar in a predetermined form with a known GL 210 and the previously measured metabolism by the patient of the known quality sugar in a predetermined form with a known GL 202 is used at least in part by the controller to adjust insulin levels during the treatment. The controller starts the infusion of insulin 214, controller starts the infusion of one or more cocktail components 216 and the sensors 110 measure the patient's blood glucose levels and measures vital signs 218.

A monitoring module 250 receives the measurements and the controller decisions if threshold levels are met for one or more of blood glucose levels (BGL) 262, oxygen 264 and vitals 266. If all threshold levels being monitored are met the then the controller continues the insulin infusion, and the system goes on to the timer module 275. If the BGL threshold 262 is not met then the controller will one or more of adjust insulin infusion, add glucose and alert via an alarm. If the O₂ threshold 264 is not met then the controller will one or more of the oxygen flow rate 150, adjust gaseous flow rate 160 of the O₂ delivered to patient, adjust one or more cocktail components 130, adjust infusion of one or more supplement/adjuvant components 140 flow rates and alert via an alarm. If the vitals threshold 266 is not met then the controller will one or more of adjust insulin infusion, adjust one or more of the cocktail and/or adjuvant infusion flow rates, add glucose and alert via an alarm. If threshold were not met then the sensors 110 measurements of the patient's blood glucose levels and measure of vital signs 218 are processed by the controller and the controller in the monitoring module 250 as described above decisions if the threshold are been met the monitoring and adjustments repeat. If threshold levels were met and insulin was continued then the system controller goes to the timer module 275. First elapsed infusion time is measured 278, if the time threshold is not met the measurements of one or more of BGL, O₂ saturation and vital signs are taken 280 and the controller in monitoring mode 250 processes the measurements and repeats the cycle. If the timer has met the threshold 300 the infusion of insulin is stopped and the infusion of cocktail components and/or adjuvant components 302 is stopped and the system measures one or more of BGL, O₂ saturation and vital signs 310 and the controller in timer mode 250 processes the measurements 350 to determine if the post insulin levels of one or more of BGL, O₂ saturation and vital signs are met. If “yes”, then the system stops. If “no” the system one or more of activates alarm and administers one or more of O₂ and glucose to the patient.

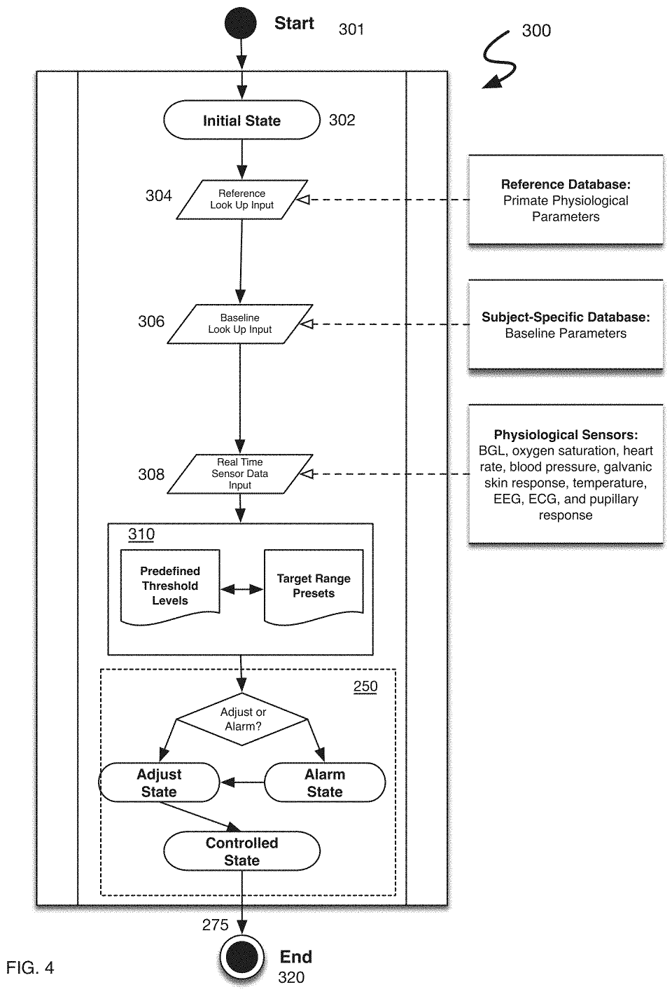

FIG. 4 illustrates aspect of controller control logic for a system and method to control hypoglycemic conditions in a primate. It is an overview of some aspects of the operational flow of the control system 300. After the start 301 the controller is in the initial state 302 and will input data from at least on LUT and when available will input data from the primate's measured baseline 306. The input data is compared with real time sensor inputs 308 and analyzed against predefined threshold or limits and/or target BGL range setting 310. The analysis continues in the monitoring module 250 (described in detail with reference to FIG. 3) wherein the control can adjust the state of the primate by adjusting one or more of glucose, insulin, oxygen, and cocktail components being administered to main or keep the primate in the controlled state (above threshold levels and within target range) and then enter timer module 275 if the timer has timed out end 320.

### EXAMPLES

Cocktail agents for treating abnormal cells although described herein as being targeted to abnormal cancerous cells and senescent cells there is not always a bright line and due to the connection between some senescent cells and cancerous cells some of the senolytic therapeutics have efficacy in treating abnormal cells in the cancerous group. However, in all instances it is the use of controlled hypoglycemic conditions which support the use of lower toxicity cocktails. Lower toxicity refers to toxicity to normal cells compared to abnormal cells. Examples listed herein are not intended to be limiting. But rather, a solution of the disclosed delivery system is that the controlled induced hypoglycemic state improves delivery rates of cocktail and/or adjuvants to the abnormal cells at a higher rate than the normal cells. In some instances, the delivery under hypoglycemic conditions improves bioavailability of compounds which are more toxic to abnormal cells than normal cells. The increased utilization of the cocktail compounds by the abnormal cells can reduce the toxicity to normal cells by allowing for a lower dose.

Exemplars of cocktail agent for treating abnormal cells include but are not limited to artemisia (and sesquiterpene lactones found therein), hydrogen, oxyhydrogen, vitamin C, curcuminoid, cannabinoids, γ-Tocotrienols. Broad groups of chemotherapeutic agents include, but are not limited to, alkylating agent, plant alkaloids, antitumor antibiotics, antimetabolites, topoisomerase inhibitors, mitotic inhibitors, nitrosoureas and anthracyclines. Senolytics, which may also have efficacy in treating abnormal cancerous cells, include azithromycin, Co-enzyme Q10, quercetin (and analogs thereof), enzastaurin, dasatinib, Sirolimus (Rapamycin), Nay-Gal and Navitoclax.

Senolytics are a class of drugs that selectively clear senescent cells (SC). However, in some instances they also address cancer. For example, histone deacetylase (HDAC) inhibitors can act as senolytics and have also been used in treating cancer. They are anti-cancer agents that block the function of an enzyme called HDAC, allowing the expression of genes that are involved in cell division and ultimately slowing down the spread of cancer. Specific HDAC inhibitors include, but are not limited to, romidepsin, zolinza (vorinostat), belinostat (also known as PXD101), farydak, panobinostat), ricolinostat (also known as ACY-1215) and citarinostat (also known as ACY-241).

### Examples of Cocktail or Adjuvant Components

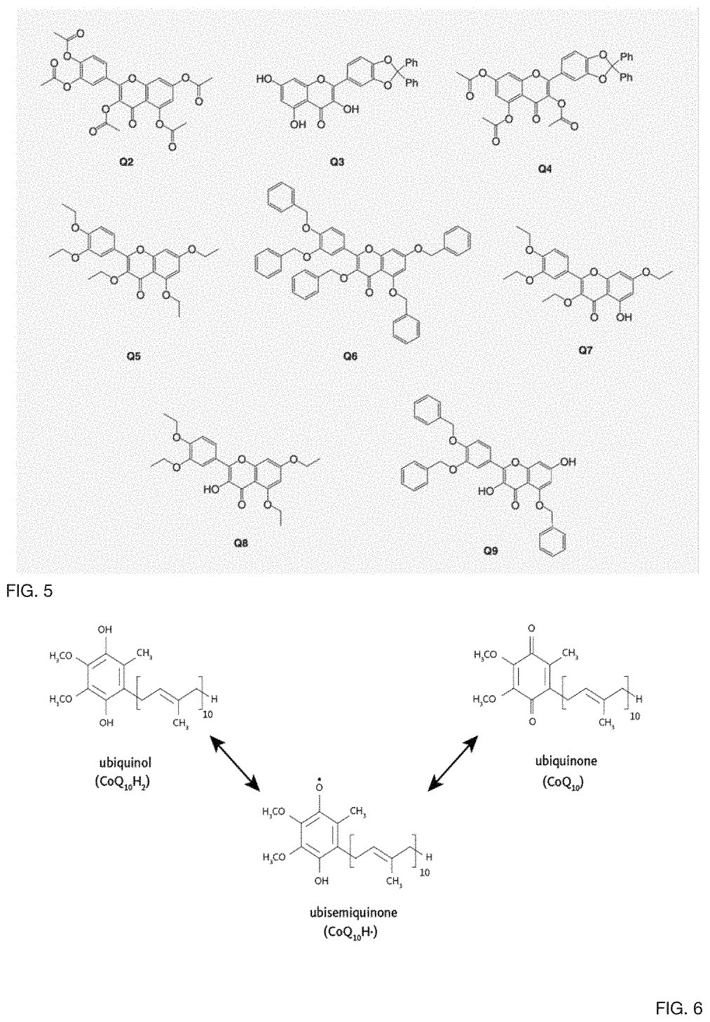

Quercetin is poorly soluble in water and unstable in physiological systems, and its bioavailability is very low. Quercetin is a flavonoid widely present in plants and has demonstrated pharmacological properties, including anticancer and senolytic. Quercetin analogs have been studied to increase the low availability of Quercetin, see FIG. 5. Quercetin has also been shown to display senolytic effects in some primary senescent cells. (Pleiotropic Effects of Tocotrienols and Quercetin on Cellular Senescence: Introducing the Perspective of Senolytic Effects of Phytochemicals. Marco Malavolta, Elisa Pierpaoli, Robertina Giacconi, Laura Costarelli, Francesco Piacenza, Andrea Basso, Maurizio Cardelli, Mauro Provinciali. Current Drug Targets Volume 17, Issue 4, 2016 DOI: 10.2174/1389450116666150907105104)

Co-enzyme Q10 can act as a senolytic. There are three redox states of CoQ:10 fully oxidized (ubiquinone), semiquinone (ubisemiquinone), and fully reduced (ubiquinol). See FIG. 5. Deficiencies on Co-enzyme Q10 have been shown to be prevalent in cancer patients. Accordingly, in some instances the optional addition of Q10 as an adjuvant when treating abnormal cells is appropriate.

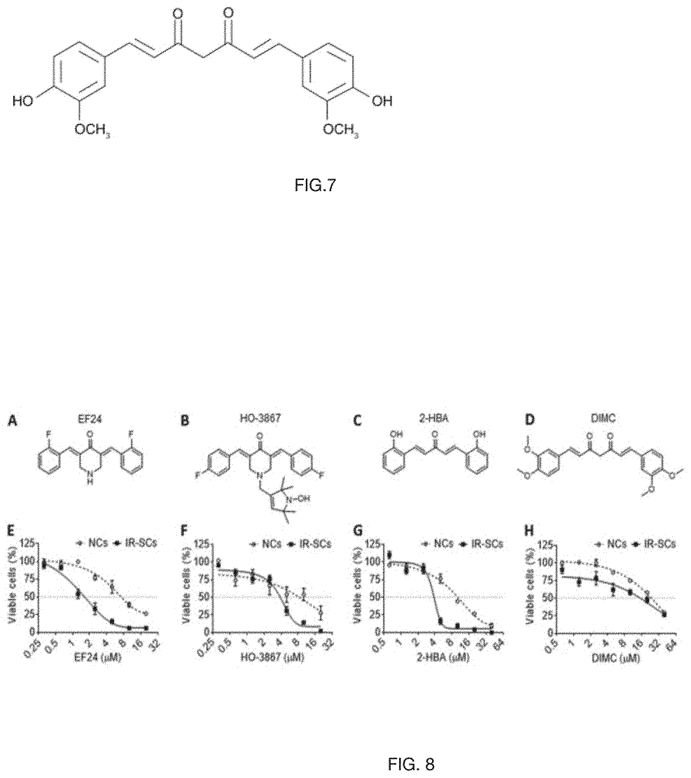

Curcumin has been shown to have both senolytic and anticancer properties. Curcumin is a natural compound extracted from the turmeric (see FIG. 7). Numerous studies suggest that curcumin has some health benefits in treating age-related diseases. Curcumin has been shown to prolong lifespan and extend health span in Drosophila melanogaster (fruit fly) and Caenorhabditis elegans. Administration of curcumin is difficult due to its low aqueous solubility, poor oral bioavailability, and rapid degradation under physiological conditions.

To improve the bioavailability of curcumin, curcumin analogs have been developed, including EF24, HO-3867, 2-HBA and dimethoxycurcumin (see FIG. 8). One analog of Curcumin is EF24, it is reported as being 10-fold more potent in inducing cancer cell death than curcumin. EF24 is reported as more efficacious against various cancer cells but less toxic to normal cells. Other representative curcumin analogs, including HO-3867, 2-HBA and DIMC, were also reported to have better anti-cancer activities than curcumin. One study identified EF24 as a potent and broad-spectrum senolytic agent. They found that EF24 can selectively decrease the cell viability in a variety of senescent cells (see Li W, He Y, Zhang R, Zheng G, Zhou D. The curcumin analog EF24 is a senolytic agent. Aging (Albany NY). 2019; 11(2):771-782. doi:10.18632/aging.101787)).

As an anticancer agent Curcuminoids suffer from low bioavailability, combining them with micelles or liposomes may ameliorate some of this low availability or by adding adjuvants. Curcuminoids have been shown to exerted anticancer properties in vitro, ex vivo and in vivo as well as in clinical trials by regulating a variety of biological pathways involved in tumor invasion, metastasis, and angiogenesis (https://www.frontiersin.org/article/10.3389/fchem.2014.00113).

A number of schema have been investigated in the art to improve curcumin bioavailability. Curcumin may be solubilized in a number of ways including but not limited to using a solvent such as DMSO or ethanol, loading curcumin into one of a micelle, nanoparticle, or liposome.

In some instances, we have included 500 mg of curcumin in a 50 ml aqueous solution with 95% total curcuminoid content having 71% curcumin, in DMSO with Kolliphor HS 15 (also known as Macrogol 15 Hydroxystearate, Polyoxyl 15 Hydroxystearate) sodium citrate use only after dilution at least 1:10.

The hydrophobic nature of curcumin presents challenges for bioavailability. A liposome with a mean particle size of about 200 nms composed of dipalmitoylphosphatidylcholine (Lipoid GMBH, Germany) and cholesterol (Carbogen 134 Amcis B. V., The Netherlands) acts as a vehicle to deliver the curcumin at between about 250 mg and about 500 mg. In some instances, the curcumin may be ingested. In other instances, curcumin may be intravenously administered and can also be provided with a hydrophilic carrier.

It has also been reported that EF24 can synergistically kill senescent cells when combined with Navitoclax.

In some instances, Bee propolis may be added as an adjuvant or chemotherapeutic agent. Bee Propolis and its components have been shown to display strong anti-proliferative activity via suppression of proliferating cell nuclear antigen and vascular cell adhesion molecule 1 in human prostate PC-3 cancer cells.

Tocotrienols can act as senolytics. The vitamin E family comprise four tocotrienols (alpha, beta, gamma, delta) and four tocopherols (alpha, beta, gamma, delta). The critical chemical structural difference between tocotrienols and tocopherols is that tocotrienols have unsaturated isoprenoid side chains with three carbon-carbon double bonds versus saturated side chains for tocopherols

γ-Tocotrienols isomer is a potent and specific inhibitor of prostate cancer (Pica) cell proliferation and invasion which acts through multiple molecular pathways. γ-Tocotrienols may be used alone or in combination with chemotherapy for treating advanced stage PCa. (Yap W N, Chang P N, Han H Y, et al. Gamma-tocotrienol suppresses prostate cancer cell proliferation and invasion through multiple-signaling pathways. Br J Cancer. 2008; 99(11):1832-1841. doi: 10.1038/sj.bjc.6604763).

Disclosed herein are compositions containing at least one of Roxithromycin and Azithromycin in a therapeutic aliquot (substantially less than 100 μM) to selectively eliminated abnormal senescent cells via autophagy. In some instances, the therapeutic aliquot is any amount between 1% and 99% 100 μM. In some instances, the therapeutic aliquot is any amount between 1% and 50% 100 μM. In some instances, the therapeutic aliquot is any amount between 1% and 25% 100 μM. In some instances, the therapeutic aliquot is any amount between 1% and 10% 100 μM. In some instances, the therapeutic aliquot is any amount less than 1% of 100 μM.

Autophagic cells have an increased likelihood of becoming senescent. This is called the autophagy-senescence transition (AST). During autophagy, cells accumulate autophagic organelles (lysosomes and auto-phagosomes) and these structures normally sequester dangerous proteases, including the cathepsins (B, S and L). However, during an acute stress, lysosomes in autophagic cells can become “leaky”, resulting in the release of the cathepsins into the cytosol, secondary to lysosome rupture or defects in the lysosomal membrane. Once in the cytosol, the cathepsins proteolytically cleave the sirtuins, such as SIRT1, and the senescence pheno-type is promoted. Disclosed herein is a method of treatment and composition which selectively tend towards autophagic and senescent cells to promote cell death of those cells.

The disclosure includes the containment of said senolytic compounds within micelles. Those of ordinary skill in the art will understand that micelles are represented by a broad spectrum of delivery packages and the disclosure is not limited to a particular micelle structure.

Azithromycin preferentially targets senescent cells. Roxithromycin and Azithromycin selectively eliminated large numbers of senescent cells at 100 μM, Azithromycin was found to be the most selective compound, as it eliminated senescent cells, without affecting control cells. (Ozsvari B, Nuttall J R, Sotgia F, Lisanti M P. Azithromycin and Roxithromycin define a new family of “senolytic” drugs that target senescent human fibroblasts. Aging (Albany NY). 2018; 10(11):3294-3307. doi:10.18632/aging.101633).

Navitoclax (also known as ABT263) works by inhibiting the Bcl-2 (B-cell lymphoma 2) pathway. The Bcl-2 gene is the founding gene of the Bcl-2 family of proteins that regulate cell death, by either inhibiting or inducing apoptosis. navitoclax (ABT 263) In vitro and in vivo studies of Nav-Gal has shown a high potency of this prodrug to mitigate tumor progression Navitoclax with a galacto-conjugation is referred to as (Nav-Gal), which aims to minimize the platelet toxicity effect and increase the selectivity toward tumors-accumulating senescent cells (González-Gualda, E., Páez-Ribes, M., Lozano-Torres, B., Macias, D., Wilson, J. R., González-López, C., et al. (2020). Galacto-conjugation of Navitoclax as an efficient strategy to increase senolytic specificity and reduce platelet toxicity. Aging Cell 19 (4), e13142. doi:10.1111/ace1.13142). Venetoclax (ABT-199), another of these new agents, is a highly selective oral inhibitor of the bcl-2 anti-apoptotic pathway.

Dasatinib (D), which has been approved for clinical use in the United States since 2006, and Quercetin (Q), the naturally occurring flavonoid that makes apple peels taste bitter and have been shown in animal models to reduce abnormal cell populations. Additional senolytic drugs are discussed in the following paper. Kirkland J L, Tchkonia T. Senolytic drugs: from discovery to translation. J Intern Med. 2020; 288(5):518-536. doi:10.1111/joim.13141

Over 100 cannabinoids have been identified in Cannabis sativa. Cannabinoids and the mammalian endocannabinoid system show immune-modulatory role of cannabinoids.

Compounds in cannabis, include delta-9-tetrahydrocannabinol (Δ9-THC), is primarily responsible for the psychoactive effects of cannabis. Delta-8-THC (delta-8-tetrahydrocannabinol) has a lower psychotropic potency and has been found to connect to both CB1 and CB2. THCA (tetrahydrocannabinolic acid) is a non-psychotropic cannabinoid found in cannabis. It is a precursor of tetrahydrocannabinol (THC) and has shown anti-inflammatory and anticancer properties (https://www.openaccessgovernment.org/ms-and-prostate-cancer/79532/).

In some instances, cannabinoids may be administered via vaporization at elevated temperatures. Cannabis flower or extracts contain a subset of the cannabinoids and terpenes with the flower are easily vaporized with heat via one of conduction and convection heating system such as those disclosed in pending U.S. patent application Ser. Nos. 16/118,244 and 16/410,858. In some instances, cannabinoids may be ingested. In other instances, cannabinoids may be intravenously administered and can also be provided with a hydrophilic carrier.

Artemisia is a herbs and shrubs which belongs to the family Compositae (Asteraceae). Within this family, Artemisia is included in the tribe Anthemideae and comprises over 500 species, which are mainly found in Asia, Europe, and North America.

Studies of artemisinins in in-vitro experiments and animal models have demonstrated broad anti-cancer activity including pro-apoptotic, anti-proliferative, anti-angiogenesis and anti-metastatic effects. Artesunate displays cytotoxic effects against numerous cancer cell lines including colon, breast, leukaemia, melanoma, central nervous system, ovarian, renal, and prostate cancers. (Efferth et al., 2003; Efferth et al., 2004; Efferth, Dunstan, Sauerbrey, Miyachi, & Chitambar, 2001; Efferth, Giaisi, Merling, Krammer, & Li-Weber, 2007; Nunes, Pandey, Yadav, Goel, & Ateeq, 2017). The active metabolite of artemisinins, dihydroartemisinin (DHA), has demonstrated antineoplastic effects in breast, glioma, colon, lung, ovarian, pancreatic, renal cell, and leukaemia cancer cell lines (Chauhan, Min, & Kwon, 2017; Chen et al., 2017; Chen, Li, Zhang, & Wang, 2009; Hooft van Huijsduijnen et al., 2013; Kim et al., 2006; Kumar et al., 2017; Lu, Chen, Zhang, Ding, & Meng, 2011; Mu et al., 2007; Raza, Ghoshal, Chockalingam, & Ghosh, 2017; Singh & Lai, 2001; Wang et al., 2017).

The antimalarial drug artemisinin and its derivatives have been explored as potential anticancer agents. In one study, it was found that artemisinin compounds can sensitize cancer cells to ferroptosis (programmed cell death driven by iron-dependent lipid peroxidation). Dihydroartemisinin (DAT) can induce lysosomal degradation of ferritin in an autophagy-independent manner, increasing the cellular free iron level and causing cells to become more sensitive to ferroptosis. (Augustin Y, Staines H M, Krishna S. Artemisinins as a novel anti-cancer therapy: Targeting a global cancer pandemic through drug repurposing. Pharmacol Ther. 2020; 216:107706. doi: 10.1016/j.pharmthera.2020.107706) The transferrin receptors TfR2 and TfR1 are membrane receptors which mediate cellular uptake of iron from plasma glycoprotein, transferrin. Iron uptake from transferrin involves the binding of transferrin to the transferrin receptor, internalization of transferrin within an endocytic vesicle by receptor-mediated endocytosis and the release of iron from the protein by a decrease in endosomal pH. TfR2 is frequently expressed in tumor cell lines. Particularly frequent was its expression in ovarian cancer, colon cancer and glioblastoma cell lines. The major iron transporters belong to the transferrin family including transferrin (Tf), melanotransferrin (MTf), and lactoferrin (Lf). Three membrane iron transporters have been identified DMT1, Zrt-, and Irt-like protein 14 (ZIP14) and zinc transporter ZIP8 (ZIP8). TFR1 is widely overexpressed in cancers (Shen Y, Li X, Dong D, Zhang B, Xue Y, Shang P. Transferrin receptor 1 in cancer: a new sight for cancer therapy. Am J Cancer Res. 2018; 8(6):916-931. Published 2018 Jun. 1.) Controlled hypoglycemic infusion of transferrin conjugated carriers of chemotherapeutic compounds will be metabolized at a higher rate in cancer cells then normal cells due to the overexpression of TFR in such abnormal cells. However, targeting with chemotherapeutic agents can be blocked or diminished by native transferrin in the blood which may interfere with the effects of these transferrin conjugates leading to decreased therapeutic efficacy. By utilizing hypoglycemic conditions abnormal cells and in particular cancer cell membranes are destabilized and intake cocktail components both conjugated with transferrin (or a complex) and non-conjugated at a higher rate via both the glucose pathways and the TFR pathways.

Animal models have shown that to improve the tumor-targeting properties of cisplatin, transferrin has been used as a carrier to transfer cisplatin into cancer cells via transferrin receptor 1 (TfR1). The binding ability of transferrin (Tf) conjugated cisplatin can be improved by controlled hypoglycemic conditions as described herein. In a tumor-bearing mouse model, a Tf-cisplatin complex inhibited tumor growth in vivo more effectively than free cisplatin, with less toxicity in other tissues. (Peng H., Jin H., Zhuo H., Huang H. Enhanced antitumor efficacy of cisplatin for treating ovarian cancer in vitro and in vivo via transferrin binding. Oncotarget. 2017; 8: 45597-45611. Retrieved from https://www.oncotarget.com/article/17316/text/). Accordingly, any chemotherapeutic agent or senolytic which can be conjugated with transferrin may be part of the cocktail to leverage TfR pathways. Preferably the conjugated agent is less toxic to normal cells then to abnormal cells.

Talizumab a monoclonal antibody has recently shown good trial results in for prostate cancer in men with BRCA mutations and PALB2 or ATM mutations. Under hypoglycemic conditions a higher or large quantity of Talizumab will pass into the prostate cancer cell and act to disrupt their DNA repair and therefore promote apoptosis under such hypoglycemic conditions.

Vitamin C is an antioxidant with immunomodulatory properties and significantly concentrates in important immune cells. High doe's IV vitamin C between 5-50 g (in exceptional cases up to 60 and even to 120 g/day) in combination with chemotherapeutic agents has been shown to have therapeutic advantages against breast cancer cells. Lee S J, Jeong J H, Lee I H, Lee J, Jung J H, Park H Y, Lee D H, Chae Y S. Effect of High-dose Vitamin C Combined with Anti-cancer Treatment on Breast Cancer Cells. Anticancer Res. 2019 February; 39(2):751-758. doi: 10.21873/anticanres.13172. PMID: 30711954.

Molecular hydrogen has been shown to be an anti-inflammatory. H₂ has been shown to decrease the expression of a number of pro-inflammatory factors, including at least tumor necrosis factor-α (TNF-α), interleukin (IL)-6, IL-1β, IL-10, IL-12, chemokine ligand 2 (CCL2. Furthermore, H₂-rich saline reduced serum diamine oxidase, TNF-α, IL-1β, IL-6, tissue malondialdehyde, protein carbonyl and myeloperoxidase activity, and also inhibited pro-apoptotic players, including JNK and caspase-3.

It has been reported that hydrogen gas inhalation has significantly reduced the number of total cells, eosinophils, and lymphocytes in the bronchial alveolar lavage fluid, and increased the level of IL-4, IL-13, TNF-α and chemokine (C-X-C motif) ligand 15. The IL-4 serum level was significantly decreased following inhalation. H₂ gas inhalation markedly upregulated the activity of superoxide dismutase and significantly attenuated the increased level of malondialdehyde and myeloperoxidase in allergic asthmatic mice (see Zhang N, Deng C, Zhang X, Zhang J and Bai C: Inhalation of hydrogen gas attenuates airway inflammation and oxidative stress in allergic asthmatic mice. Asthma Res Pract 4: 3, 2018). Hydrogen gas has been shown in animal models to reduce concentrations of IL-4, IL-13 and TNF-α. The efficacy of molecular hydrogen on cancer have been reported in several types of tumors including skin squamous cell, carcinomas lung cancer, ovarian cancer, thymic lymphoma, liver tumors, renal cell carcinoma, colon cancer and glioblastoma (GBM). Hydrogen as an anti-inflammatory has been shown to reduce the adverse effects of chemotherapeutic agents on the patient by reducing at least inflammation. In combination with the aqueous portion of the cocktails described herein the controller or controllers 102 in signal communication with a processor controls the administration of molecular hydrogen gas and/or as oxyhydrogen 50 during treatment to at least one of reduce inflammation, reduce the adverse effects of the chemotherapeutic agents and as an additional or adjuvant therapeutic to treat the abnormal cancerous cells.

Treating Glioblastoma (GBM) we have found that administration of hydrogen via oxyhydrogen gas suppresses inflammation and tumor progression. Administering the oxyhydrogen or molecular hydrogen gas during the controlled hypoglycemic process should improve efficacy as the tumor cells will have more permeable cellular membranes.

We have applied a cocktail approach during hypoglycemic conditions of sequenced pharmaceutically effective doses of cocktail compounds to treat cancer. Treatment includes IV administration of 0.1-0.3 IU Insulin/kg bodyweight followed by a sequenced IV administration of multiple chemotherapeutic agents based on the recommendations listed in the oncologic guidelines.

The amount of insulin administered can be a function of pretreatment. When a patient consumes a known quality sugar in a predetermined form with a known glycemic Index (GI) and a known glycemic load (GL) measurement of the patient's innate systems response to the consumed material can be measured via blood glucose monitoring. Thereafter the controller uses the previously acquired measurements to set target or threshold blood glucose levels to adjust for during controlled hypoglycemic treatment. In operation the patient consumes the same known quality sugar in a predetermined form with a known glycemic Index (GI) and a known glycemic load (GL) at a predetermined time before the controlled hypoglycemic treatment. This system and method personalize the hypoglycemic process to an individual thereby reducing the risk of insulin shock and seizure.

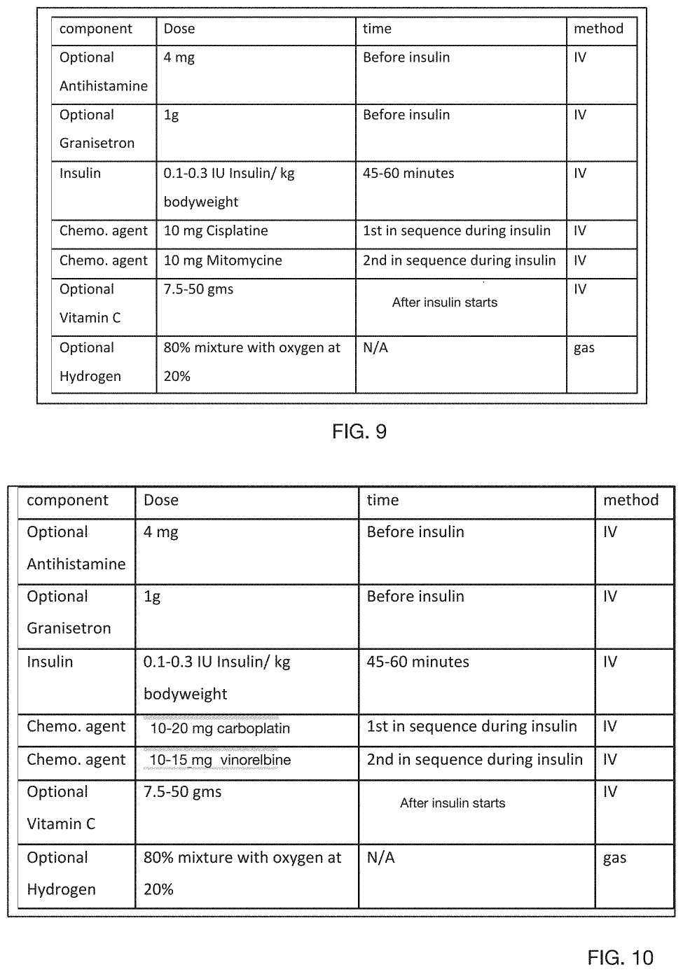

In general, with our system when the blood glucose level is dropping in a controlled and monitored fashion we are able to have efficacious treatment with approximately 5-10% of the recommended dose of chemo over the same course of 45-60 minutes. The system disclosed herein is configured to sample the sensor data to closely monitor the patient. Before, during and after the system monitors hypoglycemic inducement measuring at least one of EKG, EEG, heart rate, blood pressure, oxygen saturation, glucose levels, pupillary response, temperature, electro galvanic skin resistance/response. The Galvanic Skin Response (GSR), also named Electrodermal Activity (EDA) and Skin Conductance (SC), is the measure of the continuous variations in the electrical characteristics of the skin, i.e., for instance the conductance, caused by the variation of the human body sweating. Sweating is correlated to the effect of the insulin and an indication of treatment progression and status. Optionally at least one of Vitamin C may be added to the IV, hydrogen gas, oxyhydrogen may be administered during or following the controlled hypoglycemic condition. Although not listed in the flowing tables additional supplements and adjuvants may be included in the cocktail. We have observed in several different cancerous tumors that we can utilize less chemotherapeutic agents in the cocktail under hypoglycemic conditions then would be efficacious without hypoglycemic conditions. FIGS. 9-15 disclose tables showing sequenced administration of hypoglycemic condition and infusion of therapeutic compounds.

For lung cancer: small cell diagnosis post controlled hypoglycemic treatment there was no detachable primary tumor after 6 month. For pancreatic cancer post controlled hypoglycemic treatment there was no detachable tumor for 18 months since treatments. For ovarian Cancer patients diagnosed over 36 months ago and although the cancer had metastasized into Liver, and Lymph nodes post controlled hypoglycemic treatment there was no detachable tumor or further metastases.

As a method, composition of matter, device, and system for treating bladder cancer we have used Cisplatine and Mitomycine at levels set forth in the table shown in FIG. 9. Those of ordinary skill in the art will recognize that chemotherapeutic agents which are in the same class as Cisplatine and Mitomycine may be substituted and expect similar results under said hypoglycemic conditions.

As a method, composition of matter, device, and system for treating breast cancer we have used carboplatin combined with vinorelbine at levels set forth in the table shown in FIG. 10. Those of ordinary skill in the art will recognize that chemotherapeutic agents which are in the same class as carboplatin and vinorelbine be substitute and expect similar results under said hypoglycemic conditions.

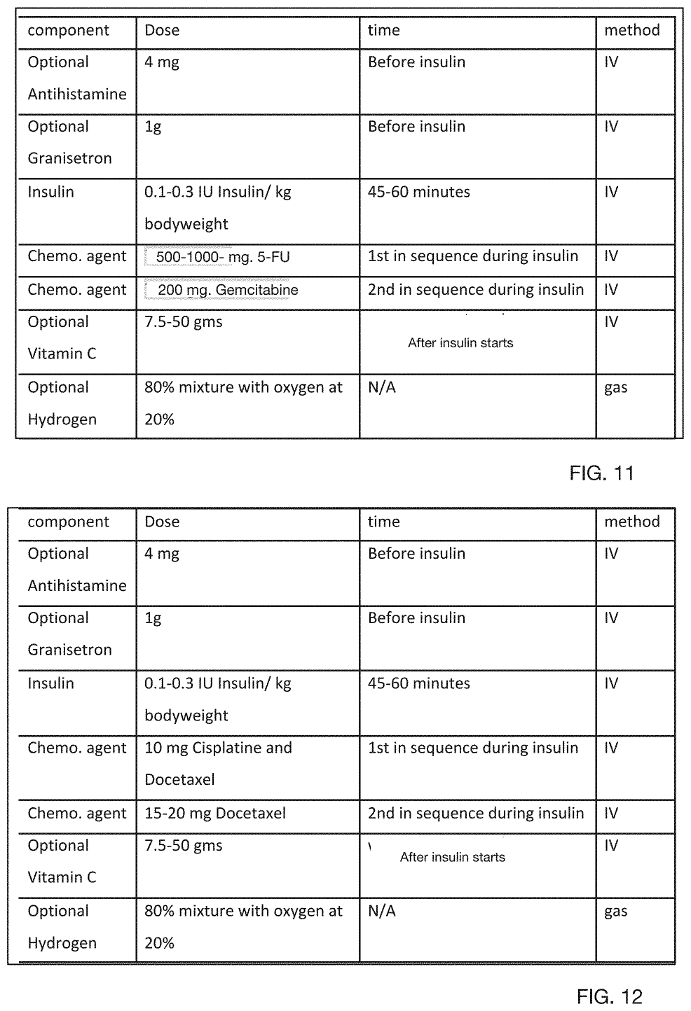

As a method, composition of matter, device, and system for treating abdominal cancer we have used 5-FU and Gemcitabine at levels set forth in the table shown in FIG. 11. Those of ordinary skill in the art will recognize that chemotherapeutic agents which are in the same class as 5-FU and Gemcitabine may be substituted and expect similar results under said hypoglycemic conditions.

As a method, composition of matter, device, and system for treating lung cancer we have used Cisplatine, and Docetaxel set forth in the table shown in FIG. 12. Those of ordinary skill in the art will recognize that chemotherapeutic agents which are in the same class as Cisplatine, and Docetaxel be substitute and expect similar results under said hypoglycemic conditions.

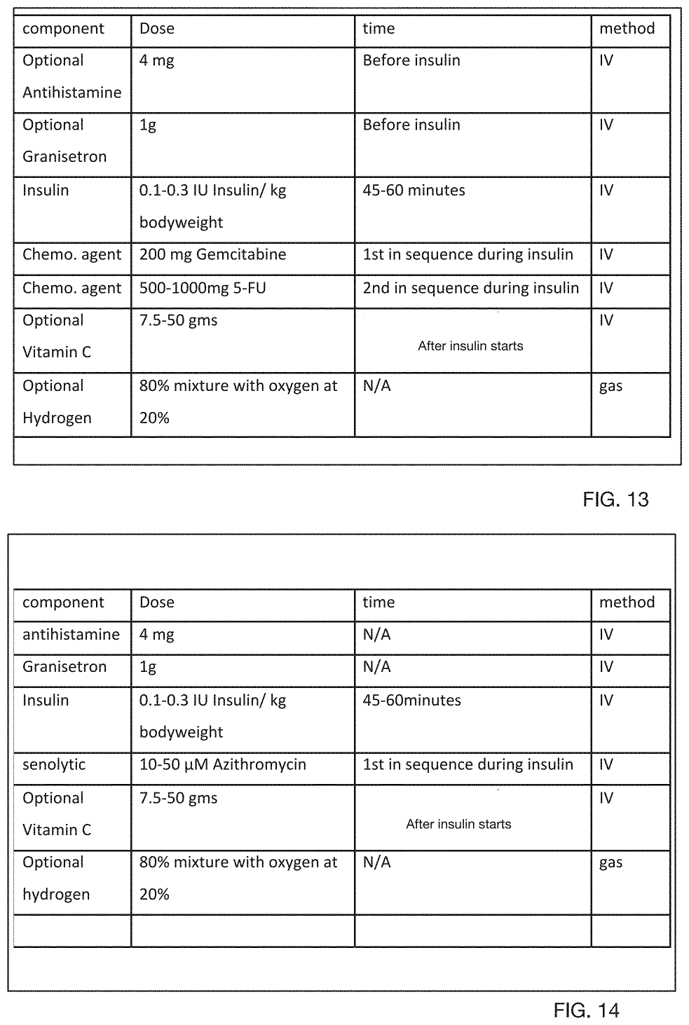

As a method, composition of matter, device, and system for treating colon cancer we have used Gemcitabine and 5-FU set forth in the table shown in FIG. 13. Those of ordinary skill in the art will recognize that chemotherapeutic agents which are in the same class as Gemcitabine and 5-FU be substituted and expect similar results under said hypoglycemic conditions.

As a method, composition of matter, device, and system for reducing senescent cell populations administering Azithromycin, during controlled hypoglycemic conditions as set forth in the table shown in FIG. 14 is disclosed.

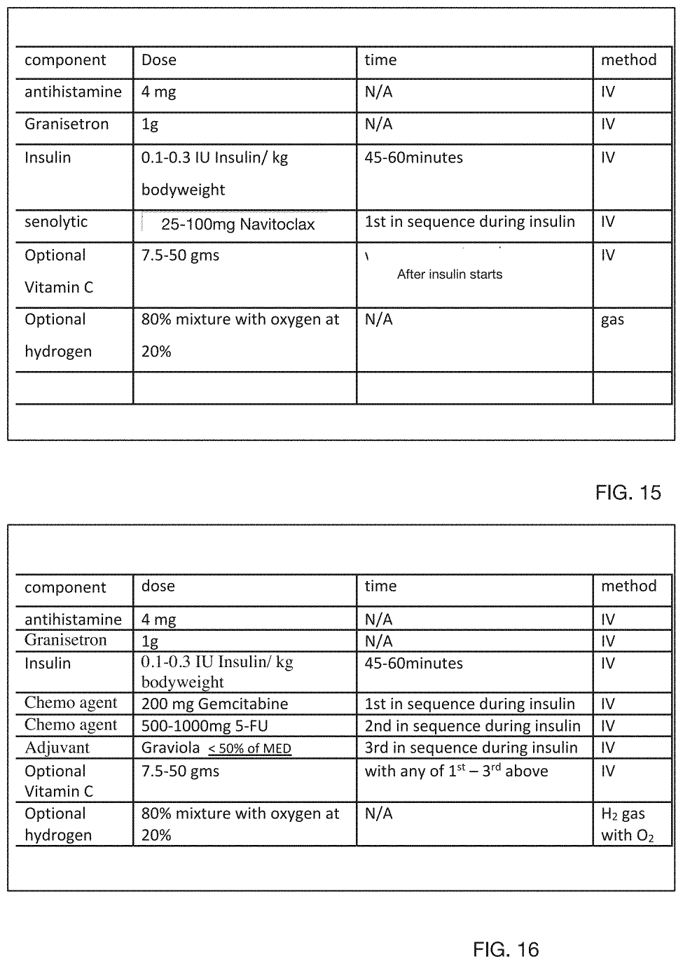

As a method, composition of matter, device, and system for reducing senescent cell populations administering Navitoclax or Nav-Gel, during controlled hypoglycemic conditions as set forth in the table shown in FIG. 15 is disclosed. Navitoclax has been test and shown to have some efficacy when combined with at least one of masitinib, rituximab, bandmaster, erlotinib and paclitaxel. (see https://www.frontiersin.org/article/10.3389/fphar.2020.564108).

Nav-Gal enhances the cytotoxicity of standard senescence-inducing chemotherapy (cisplatin) in human A549 lung cancer cells. Concomitant treatment with cisplatin and Nav-Gal in vivo results in the eradication of senescent lung cancer cells and significantly reduces tumor growth. González-Gualda E, Pàez-Ribes M. Lozano-Torres B, Macias D, Wilson J R 3rd, González-López C, Our H L, Mirón-Barroso S, Zhang Z, Lérida-Viso A, Blandez J F, Bernardos A, Sancenón F, Rovira M, Fruk L, Martins C P, Serrano M, Doherty G J, Martinez-Máñez R, Muñoz-Espín D. Galacto-conjugation of Navitoclax as an efficient strategy to increase senolytic specificity and reduce platelet toxicity. Aging Cell. 2020 April; 19(4):e13142. doi: 10.1111/ace1.13142. Epub 2020 Mar. 31. PMID: 32233024; PMCJD: PMC7189993. For use in hypoglycemic primate treatment dosage should be 50% or less of non-hypoglycemic dose for said primate.

As a method, composition of matter, device, and system for reducing senescent cell populations administering Azithromycin, during controlled hypoglycemic conditions as set forth in the table shown in FIG. 14 is disclosed.

As a method, composition of matter, device, and system for reducing senescent cell populations administering Navitoclax or Nav-Gel, during controlled hypoglycemic conditions as set forth in the table shown in FIG. 15 is disclosed.

As a method, composition of matter, device, and system for treating at least pancreatic and liver cancer cocktail components are set forth in the table shown in FIG. 16. Those of ordinary skill in the art will recognize that chemotherapeutic agents which are in the same class as Gemcitabine and 5-FU be substituted and expect similar results under said hypoglycemic conditions.

The examples of treatment in tables presented are not intended to be limiting and are nonlimiting examples of a few of the variety of cocktail components, including those that are toxic at the normally used dosages for treating abnormal cells. The exemplary implementations disclosed herein which can be used in provide for greater efficacy of cocktail components at low dose. In some instances, a cocktail component which can be toxic to a patient at the full recommended effective dosage are administered at less than 50% of the normal effective dosage and retain efficacy. In some instances, a cocktail component which can be toxic to a patient at the full recommended effective dosage can be administered at less than 40% of the normal effective dosage and retain efficacy. In some instances, a cocktail component which can be toxic to a patient at the full recommended effective dosage can be administered at less than 30% of the normal effective dosage and retain efficacy. In some instances, a cocktail component which can be toxic to a patient at the full recommended effective dosage can be administered at less than 20% of the normal effective dosage and retain efficacy. In some instances, a cocktail component which can be toxic to a patient at the full recommended effective dosage can be administered at 10% of the normal effective dosage and retain efficacy.

While the compositions and method have been described in terms of what are presently considered to be the most practical and preferred implementations, it is to be understood that the disclosure need not be limited to the disclosed implementations. It will be understood that various omissions and substitutions and changes in the form and details of the devices illustrated, and in their operation, may be made by those skilled in the art without departing from the spirit of the invention. For example, it is expressly intended that all combinations of those elements and/or method steps which perform substantially the same function in substantially the same way to achieve the same results are within the scope of the invention. It is the intention, therefore, to be limited only as indicated by the scope of the claims appended hereto.

We claim:

1. A system to maintain a hypoglycemic condition in a primate, within a predetermined blood glucose range, for treating abnormal cells comprising:
one or more sensors which measure an aspect that is physiological;
one or more fluid flow control devices;
insulin in a vessel in fluid communication with a fluid control device;
glucose in a vessel in fluid communication with a fluid control device;
at least one cocktail containing at least one of senolytic and chemotherapeutic components in one or more vessels each vessel in fluid communication with a fluid control device;
one or more controllers in signal communication with;
at least one microprocessor;
a blood glucose level (BGL) measuring sensor;
memory;
said one or more sensors;
one or more databases or lookup tables;
said fluid control devices; and,
wherein the controller controls the fluid control devices for at least insulin glucose, and the cocktail to keep blood glucose level (BGL) of the primate within a target hypoglycemic BGL range for the primate.

2. The system to induce a hypoglycemic condition within a predetermined blood glucose range for treating abnormal cells of claim 1, wherein the controller receives sensor data inputs and adjust the target hypoglycemic BGL range in response to sensory data received.

3. The system to induce a hypoglycemic condition within a predetermined blood glucose range for treating abnormal cells of claim 2, wherein the sensor data is BGL and one or more of oxygen saturation, heart rate, blood pressure, galvanic skin response, temperature, EEG, ECG, and pupillary response.

4. The system to induce a hypoglycemic condition within a predetermined target hypoglycemic BGL range for treating abnormal cells of claim 3, wherein the controller controls the administration of at least one of oxygen and hydrogen.

5. The system to induce a hypoglycemic condition within a predetermined blood glucose range for treating abnormal cells of claim 3, wherein the cocktail is at least one of, quercetin (and analogs thereof), enzastaurin, Q10 and curcumin, dasatinib, tocotrienols azithromycin, Sirolimus (Rapamycin), Nav-Gal, Navitoclax, artemisia, Bee Propolis, hydrogen, oxyhydrogen, vitamin C, Curcuminoid, cannabinoids, γ-Tocotrienols, romidepsin, zolinza (vorinostat), belinostat (also known as PXD101), farydak, panobinostat), ricolinostat (also known as ACY-1215) and citarinostat (also known as ACY-241).

6. The system to induce a hypoglycemic condition within a predetermined blood glucose range for treating abnormal cells of claim 3, wherein the cocktail is at least one of, Cisplatine, Mitomycine, carboplatin, vinorelbine, 5-FU, Gemcitabine, and Docetaxel.

7. The system to induce a hypoglycemic condition within a predetermined blood glucose range for treating abnormal cells of claim 3, wherein the cocktail is at least one of, plant alkaloids, antitumor antibiotics, antimetabolites, topoisomerase inhibitors, mitotic inhibitors, nitrosoureas and anthracyclines.

8. The system to induce a hypoglycemic condition within a predetermined blood glucose range for treating abnormal cells of claims 5-7, wherein the active agents in the cocktail are each less than 50% the maximum tolerated dose (MTD).

9. The system to induce a hypoglycemic condition within a predetermined blood glucose range for treating abnormal cells of claims 5-7, wherein the active agents in the cocktail are less than 50% the minimum effective dose (MED).

10. The system to induce a hypoglycemic condition within a predetermined blood glucose range for treating abnormal cells of claims 5-7, wherein the active agents in the cocktail are less than 25% the minimum effective dose (MED).

11. A method to reduce abnormal cell populations in a primate, the method comprising:
inducing a hypoglycemic condition in a primate having abnormal cells within a target BGL range by way of infusion, controlled by a controller, of at least insulin;
monitoring vital signs of the primate with one or more sensors each of which monitor a physiological aspect of the primate including at least BGL and is in signal communication with the controller;
controlling with a controller infusion of a cocktail containing at least one of senolytic and chemotherapeutic components into the primate while the primate is in a hypoglycemic condition;
wherein the controller receives data inputs form the sensors and at least in part uses that input data to one of maintain the primate's BGL within a target hypoglycemic BGL range and alter the target hypoglycemic BGL range or lower threshold for the hypoglycemic BGL based on the received sensor data; and.
wherein abnormal cells cellular membranes are made more susceptible to the influx of cocktail components by way of the hypoglycemic condition.

12. The method to reduce abnormal cell populations in a primate, of claim 11, wherein the abnormal cells are one of cancerous and senescent.

13. The method to reduce abnormal cell populations in a primate, of claim 12, wherein the abnormal cells have more insulin receptors then normal cells.

14. The method to reduce abnormal cell populations in a primate, of claim 13, wherein the abnormal cells have more transferrin receptors then normal cells.

15. The method to reduce abnormal cell populations in a primate, of claim 12, wherein the sensor data is one of BGL, oxygen saturation, heart rate, blood pressure, galvanic skin response, temperature, EEG, ECG, and pupillary response.

16. The method to reduce abnormal cell populations in a primate, of claim 12, wherein the controller controls the administration of at least one of oxygen and hydrogen.

17. The method to reduce abnormal cell populations in a primate, of claim 12, wherein the cocktail is at least one of, quercetin (and analogs thereof), enzastaurin, Q10, curcumin, dasatinib, tocotrienols azithromycin, Sirolimus (Rapamycin), Nav-Gal and Navitoclax, Artemisia, hydrogen, oxyhydrogen, vitamin C, Curcuminoid, cannabinoids, γ-Tocotrienols, romidepsin, zolinza (vorinostat), belinostat (also known as PXD101), farydak, panobinostat), ricolinostat (also known as ACY-1215) and citarinostat (also known as ACY-241).

18. The method to reduce abnormal cell populations in a primate, of claim 12, wherein the cocktail is at least one of is at least one of, Cisplatine, Mitomycine, carboplatin, vinorelbine 5-FU, Gemcitabine, Docetaxel, Bee Propolis, plant alkaloids, antitumor antibiotics, antimetabolites, topoisomerase inhibitors, mitotic inhibitors, nitrosoureas and anthracyclines.

19. The method to reduce abnormal cell populations in a primate, of claims 17 or 18, wherein the active agents in the cocktail are each less than 50% the maximum tolerated dose (MTD).

20. The method to reduce abnormal cell populations in a primate of claims 17 or 18, wherein the active agents in the cocktail are less than 50% the minimum effective dose (MED).

21. The method to reduce abnormal cell populations in a primate of claims 17 or 18, wherein the active agents in the cocktail are less than 25% the minimum effective dose (MED).

22. The method to reduce abnormal cell populations in a primate of claims 17 or 18, wherein the active agents in the cocktail are less than 15% the minimum effective dose (MED).

23. The method to reduce abnormal cell populations in a primate of claim 11, the method further comprising the controller raises BGL in the primate by way of infusion of at least glucose to maintain the target hypoglycemic BGL range for the primate.

24. The method to reduce abnormal cell populations in a primate of claim 23, the method further comprising the controller administers magnesium before or during administration of glucose.
